(19) United States
(12) Patent Application Publication
(10) Pub. No.: US 2015/0368625 A1
(43) Pub. Date: Dec. 24, 2015

(54) ARTIFICIAL SIGMA FACTORS BASED ON BISECTED T7 RNA POLYMERASE

(71) Applicant: Massachusetts Institute of Technology, Cambridge, MA (US)

(72) Inventors: Thomas H. Segall-Shapiro, Cambridge, MA (US); Christopher Voigt, Belmont, MA (US)

(73) Assignee: Massachusetts Institute of Technology, Cambridge, MA (US)

(21) Appl. No.: 14/388,190

(22) PCT Filed: Mar. 27, 2013
(86) PCT No.: PCT/US2013/034147

§ 371 Date: Sep. 25, 2014

Related U.S. Application Data
(60) Provisional application No. 61/616,882, filed on Mar. 28, 2012.
(60) Provisional application No. 61/616,175, filed on Mar. 27, 2012.

(51) Int. Cl. C12N 9/12; C12P 19/34

(52) U.S. Cl. CPC C12N 9/1247; C12P 19/34; C12Y 207/07006

(57) ABSTRACT

Aspects of the invention relate to a regulatory system that follows design principles of natural systems but creates novel synthetic biology tools using bisected polymerase proteins.

### RELATED APPLICATIONS

This application claims the benefit under 35 U.S.C. §119(e) of U.S. Provisional Application Serial No. U.S. 61/616,175, entitled “ARTIFICIAL SIGMA FACTORS BASED ON BISECTED T7 RNA POLYMERASE,” filed on Mar. 27, 2012 and U.S. Provisional Application Serial No. U.S. 61/616,882, entitled “ARTIFICIAL SIGMA FACTORS BASED ON BISECTED T7 RNA POLYMERASE,” filed on Mar. 28, 2012, the entire disclosure of each of which is herein incorporated by reference in its entirety.

### GOVERNMENT INTEREST

This invention was made with Government support under Grant No. EEC-0540879 awarded by the National Science Foundation. The Government has certain rights in this invention.

### FIELD OF THE INVENTION

The invention relates to recombinant expression of bisected proteins and their use in regulating gene expression.

### BACKGROUND OF THE INVENTION

Synthetic biology relies on regulating gene expression in predictable and programmable ways, often using components that are based on naturally occurring regulatory molecules. T7 RNA polymerase, which binds to and initiates transcription from specific promoters, is a common component of synthetic genetic circuits, at least in part due to its high specificity for its promoter sequence, allowing for orthogonal regulation, and its transferability between cell types. Bacterial RNA polymerases include an evolutionarily conserved core region and a sigma factor, which can be selected from a variety of different sigma factors that provide DNA binding specificity for the RNA polymerase, thereby directing transcription of specific genes.

### SUMMARY OF INVENTION

Described herein are novel methods and systems for constructing a control element in a genetic circuit. Such control elements can be used for programming biology with complex functions. Aspects of the invention relate to bisected proteins that mimic naturally occurring regulatory proteins such as sigma factors, but comprise parts that do not occur naturally in bacterial systems of interest, allowing them to be used orthogonally.

Aspects of the invention relate to a recombinant T7 RNA polymerase comprising a core fragment that has no RNA polymerase activity by itself and no ability to bind and/or target a promoter DNA sequence; and a sigma-like fragment that has specificity for a promoter DNA sequence, but comprises no RNA polymerase activity, wherein the sigma-like fragment binds the core fragment to form a protein complex that has RNA polymerase activity, targets a promoter DNA sequence for which the sigma-like fragment has specificity and initiates transcription of RNA from the promoter DNA sequence, and wherein the sigma-like fragment does not initiate transcription of RNA without binding to the core fragment.

In some embodiments, the T7 RNA polymerase is split at an amino acid selected from the group consisting of amino acids 67-74, 160-206, 301-302, 564-607, and 763-770 of T7 RNA polymerase into an N-terminal fragment and a C-terminal fragment. In some embodiments, the T7 RNA polymerase is split at an amino acid selected from the group consisting of amino acids 67, 179, 301, 601 and 767 of T7 RNA polymerase. In certain embodiments, the T7 RNA polymerase is split at amino acid 601 of T7 RNA polymerase.

In some embodiments, a methionine residue is added to the N-terminus of the C-terminal fragment, and optionally wherein one or more variable amino acid residues and/or one or more amino acid residues from the N-terminal fragment are added to the C-terminal fragment.

In some embodiments, the T7 polymerase is split at amino acid 601 to yield (1) a core fragment consisting of amino acids 1-601 of T7 polymerase (1:601) and (2) a sigma-like fragment consisting of a dipeptide of methionine and a variable amino acid joined to amino acids 601-883 of T7 polymerase (M X 601:883), optionally a dipeptide of methionine and a lysine joined to amino acids 601-883 of T7 polymerase (M K 601:883).

In some embodiments, the core fragment and the sigma-like fragments are each fused to heterospecific protein interaction domains (PID) that interact with each other, to form a PID-core fragment fusion and PID-sigma-like fragment fusions, and wherein the association of the core fragment and the sigma-like fragment to form the recombinant T7 polymerase is increased relative to the association of the core fragment and the sigma-like fragment without fusion to PIDs. In some embodiments, the PIDs are coiled-coil domains. In some embodiments, the coiled-coil domains are synzip coiled-coil domains. In certain embodiments, the coiled-coil domains are synzip coiled-coil domains synzip 17 and synzip 18.

In some embodiments, a flexible linker links the PIDs to the core fragment or the sigma-like fragment. In some embodiments, the flexible linkers comprise amino acids, such as 5-7 amino acids. In some embodiments, the sigma-like fragment of the recombinant T7 RNA polymerase is engineered to have a non-native promoter DNA sequence specificity.

Further aspects of the invention relate to a system comprising a core fragment that has no RNA polymerase activity by itself and no ability to bind and/or target a promoter DNA sequence; and a set of sigma-like fragments, each of which has specificity for and/or targets a promoter DNA sequence but has no RNA polymerase activity by itself; wherein each sigma-like fragment in the set of sigma-like fragments binds the core fragment to form a protein complex that has RNA polymerase activity, targets a promoter DNA sequence for which the sigma-like fragment has specificity and initiates transcription of RNA from the promoter DNA sequence, and wherein the sigma-like fragments do not initiate transcription of RNA without binding to the core fragment.

In some embodiments, the core fragment and each of the set of sigma-like fragments is a fragment of T7 RNA polymerase. In some embodiments, the T7 RNA polymerase is split at an amino acid selected from the group consisting of amino acids 67-74, 160-206, 301-302, 564-607, and 763-770 of T7 RNA polymerase into an N-terminal fragment and a C-terminal fragment. In some embodiments, the T7 RNA polymerase is split at an amino acid selected from the group consisting of amino acids 67, 179, 301, 601 and 767 of T7 RNA polymerase. In certain embodiments, the T7 RNA polymerase is split at amino acid 601 of T7 RNA polymerase.

In some embodiments, a methionine residue is added to the N-terminus of the C-terminal fragment, and optionally wherein one or more variable amino acid residues and/or one or more amino acid residues from the N-terminal fragment are added to the C-terminal fragment. In some embodiments, the T7 polymerase is split at amino acid 601 to yield (1) a core fragment consisting of amino acids 1-601 of T7 polymerase (1:601) and (2) a sigma-like fragment consisting of a dipeptide of methionine and a variable amino acid joined to amino acids 601-883 of T7 polymerase (M X 601:883), optionally a dipeptide of methionine and a lysine joined to amino acids 601-883 of T7 polymerase (M K 601:883).

In some embodiments, the core fragment and the sigma-like fragments are each fused to heterospecific protein interaction domains (PID) that interact with each other, to form a PID-core fragment fusion and PID-sigma-like fragment fusions, and wherein the association of the core fragment and the sigma-like fragments to form the protein complex is increased relative to the association of the core fragment and the sigma-like fragments without fusion to PIDs. In some embodiments, the PIDs are coiled-coil domains. In some embodiments, the coiled-coil domains are synzip coiled-coil domains. In certain embodiments, the coiled-coil domains are synzip coiled-coil domains synzip 17 and synzip 18.

In some embodiments, a flexible linker links the PIDs to the core fragment and/or the sigma-like fragments. In some embodiments, the flexible linkers comprise amino acids, such as 5-7 amino acids.

In some embodiments, each of the set of sigma-like fragments is engineered to have a different promoter DNA sequence specificity. In some embodiments, the system further comprises nucleic acids comprising promoter DNA sequences that are specifically bound by each of the set of sigma-like fragments. In some embodiments, each promoter is activated at least 10-fold more by its cognate sigma-like factor than by any non-cognate sigma-like factor.

In some embodiments, the promoter DNA sequences are operably linked to a reporter sequence and/or a protein coding sequence. In some embodiments, the core fragment and each of the set of sigma-like fragments is independently expressed. In some embodiments, the core fragment is expressed constitutively from a single copy plasmid and/or wherein each sigma-like fragment is expressed from a medium-high copy plasmid.

In some embodiments, expression of at least each of the set of sigma-like fragments is controlled by inputs to the system, optionally conditions that the system is exposed to. In some embodiments, expression of the core fragment is constitutive. In some embodiments, the system is in a cell.

Further aspects of the invention relate to methods of controlling RNA transcription of one or more DNA sequences comprising placing the one or more DNA sequences under the transcriptional control of systems described herein. In some embodiments, each of the one or more DNA sequences is operably linked to a promoter DNA sequence that is specifically bound by at least one of the set of sigma-like fragments. In some embodiments, the ratio of the expression of the set of variable proteins determines output of the system.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 presents an overview of a non-limiting embodiment of a system in which sigma factor-like transcription factors are created by splitting T7 polymerase. The activity of the split proteins in the library is demonstrate. The core fragment, also referred to as the conserved fragment, determines the overall level of transcription, while the sigma-like fragments, also referred to as variable fragments, allocate the core fragment to different promoters.

FIG. 2. shows a bisection map of T7 RNA polymerase. Specifically shown is the relative function of the in-frame functional split points found by splitting T7 RNA polymerase using a random transposon insertion method. The average relative function of 36 unique in-frame split points is shown. When more than one clone was found to be split at the same point, the values were averaged. The most active split sites in each general region of the polymerase are marked with their split position. Dashed lines indicate the region that splits were allowed to occur within, and the grey lines indicate the variable recognition loop of T7 RNA polymerase. Data shown is the mean of four independent replicates. The values from each day were normalized to an average value of 1 to account for variability in the assays.

FIG. 3 depicts a non-limiting embodiment of a MuA transposition method used for protein bisection.

FIG. 4 presents a non-limiting schematic of conserved core fragments and variable “sigma-like” fragments.

FIG. 5 demonstrates how the addition of synzip coiled coils increases the function of split T7 RNA polymerase. Split points from each of the five seams indicated in FIG. 2 were assayed for function with and without additional synzip domains. Data shown is from four technical replicates. For each of the five seams, data for “no coils” is presented on the left and “with coils” is presented on the right.

FIG. 6 shows a non-limiting embodiment of a system for testing aspects of the invention, including a generator plasmid that produces a conserved T7 fragment and determines total transcriptional units in the system. In some embodiments, the generator plasmid is present in very low copy number. FIG. 6 also demonstrates an allocator plasmid, which produces variable T7 fragments and targets transcriptional units to specific promoters. In some embodiments, the allocator plasmid is present in medium copy number. FIG. 6 also demonstrates a reporter/effector plasmid, which contains promoters that are targeted by T7 variants and a desired output. In some embodiments, the reporter/effector plasmid is present in low copy number.

FIG. 7 demonstrates saturation of a core fragment with sigma-like fragments. This figure shows the activity of the split T7 system when the core is held constant and expression of the sigma-like fragment increased. The core fragment was expressed constitutively at two expression levels by varying the ribosome binding site (RBS) used, or not expressed at all: Black squares=higher expression (RBS19), grey circles=lower expression (RBS22), white diamonds=no core expressed. The sigma-like fragment was expressed inducibly from a pTac promoter, and the x-axis values represent the approximate relative output of this promoter. As IPTG is added to the system, the T7 RNA polymerase activity increases, then remains relatively constant as the core is saturated. The level of core expressed determines the maximum amount of activity attainable. Data shown is the mean and standard deviation from three biological replicates performed on separate days.

FIG. 8 shows a graph of a set of orthogonal sigma-like fragments. Three functional, orthogonal, sigma-like fragments were engineered. The core fragment was expressed at a constant level and the three sigma-like fragments plus a negative control were combinatorially tested with the three target promoters. Each of the sigma-like factors was able to bind the core fragment and activate its target promoter by a comparable amount (the highest on-target activity was approximately 3.1 times the lowest). Additionally, the promoters only responded to their cognate sigma-like factor; each promoter was activated at least 10 times more by its cognate sigma-like factor than by any non-cognate.

FIG. 9 shows a graph of sigma-like fragment competition. When two sigma-like fragments were expressed with a constant level of the core fragment, there was a clear tradeoff in their activities. The T7 sigma-like fragment was expressed at a constant level and the T3 sigma-like fragment was expressed at varying levels. As the level of the T3 sigma-like fragment increased, activity at a T3 promoter specifically increased (white circles), while T7 specific promoter activity decreased (grey diamonds). The activity at each of these promoters is shown as a percentage of their maximum activity measured with the same amount of core fragment. The sum of these two normalized activities (black triangles) is very close to 100% over the expression range tested, indicating that the entirety of the core fragment pool is being allocated to the two sigma-like fragments.

FIG. 10 shows a non-limiting example of modeling for systems described herein and uses of such systems. In this model, A is a core fragment, while B1 and B2 are sigma-like fragments.

### DETAILED DESCRIPTION

Aspects of the invention relate to systems comprising bisected proteins, such as RNA polymerases, wherein the RNA polymerase is split into a core fragment and a sigma-like fragment. Transcriptional activity of the core fragment of the RNA polymerase is controlled by a variety of different sigma-like fragments that can bind to the core fragment and regulate its DNA binding specificity. Thus, a repertoire of orthogonal sigma-like transcriptional regulators is created.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Aspects of the invention relate to systems comprising two fragments wherein the functional abilities of the two fragments are different when they are apart than when they are bound to each other within a protein complex. When the two fragments are apart, they are not able to activate transcription but when they are bound to each other in a complex, they are able to bind to specific regions of DNA and to activate transcription at a specific DNA sequence.

Fragments within systems described herein include core fragments and sigma-like fragments. As used herein, “core fragment,” “conserved protein” and “conserved fragment” are used interchangeably to refer to a fragment of a system that confers RNA polymerase activity when bound to a sigma-like fragment. As used herein, “sigma-like fragment,” “variable protein,” and “variable fragment” are used interchangeably to refer to a fragment of a system that confers DNA-binding activity when bound to a core fragment. A system can include multiple core fragments and/or multiple sigma-like fragments. In some embodiments, a system includes one core fragment and multiple sigma-like fragments. Each sigma-like fragment can confer different DNA-binding specificity to the protein complex and the system.

Aspects of the invention relate to RNA polymerases. It should be appreciated that any RNA polymerase protein from any source, or functional fragment thereof, can be compatible with aspects of the invention. RNA polymerases can be naturally occurring or can be synthetic. In some embodiments, the RNA polymerase is T7 RNA polymerase. In some embodiments, the T7 RNA polymerase sequence is the wild-type Bacteriophage T7 RNA polymerase sequence, corresponding to GenBank identifier NP_{—}041960.1 (SEQ ID NO:1). The T7 RNA polymerase can contain one or more amino acid differences from the wild-type protein sequence. In some embodiments, the T7 RNA polymerase sequence contains a point mutation in amino acid residue R632 relative to SEQ ID NO:1. In certain embodiments, the T7 RNA polymerase sequence contains the mutation R632S relative to SEQ ID NO:1. T7 RNA polymerase containing the R632S mutation corresponds to SEQ ID NO:2. The T7 RNA polymerase sequence containing the R632S mutation is described further in, and incorporated by reference from Temme et al. (2012) Nucleic Acids Research 40(17):8773-8781. Without wishing to be bound by any theory, in some embodiments, the R632S mutation may reduce toxicity. In other embodiments, T7 RNA polymerase contains one or more mutations other than, or in addition to, the R632S mutation.

In some aspects, the core fragment and the sigma-like fragment are created by splitting or bisecting an RNA polymerase such as a T7 RNA polymerase. For example, in some embodiments, a T7 RNA polymerase is split at an amino acid selected from the group consisting of 67-74, 160-206, 301-302, 564-607 and 763-770 relative to SEQ ID NO:1 into an N-terminal fragment and a C-terminal fragment. The two fragments are the core fragment and the sigma-like fragment. In most cases, the core fragment is the N-terminal fragment, and the sigma-like fragment is the C-terminal fragment. However, when the site at which the T7 RNA polymerase is split comes after the recognition loop, such as when T7 RNA polymerase is split at an amino acid in the group 763-770 relative to SEQ ID NO:1, then the core fragment is the C-terminal fragment, and the sigma-like fragment is the N-terminal fragment. In some embodiments the T7 RNA polymerase is split at position 67, 68, 69, 70, 71, 72, 73, 74, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 301, 302, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 763, 764, 765, 766, 767, 768, 769 or 770. In certain embodiments, the T7 RNA polymerase is split at a position selected from the group consisting of positions 67, 179, 301, 601 and 767. In certain embodiments, the T7 RNA polymerase is split at amino acid residue 601. The sequence of the core fragment when the split occurs at residue 601 is provided by SEQ ID NO:3. A representative sequence for the sigma-like fragment when the split occurs at residue 601 is provided by SEQ ID NO:5, which contains residues 601-883 of T7 RNA polymerase. SEQ ID NO:11 corresponds to SEQ ID NO:5 with a methionine residue and a variable residue at the N-terminal end. SEQ ID NO:12 corresponds to SEQ ID NO:5 with a methionine residue and a lysine residue at the N-terminal end.

When the RNA polymerase, such as T7 RNA polymerase is split, one or more amino acids can be added to the C-terminal fragment and/or the N-terminal fragment. In some embodiments, one or more amino acids are added to the C-terminal fragment but no amino acids are added to the N-terminal fragment. For example, a methionine residue can be added to the C-terminal fragment. In some embodiments, one or more variable amino acids can be added following the methionine residue. As used herein, a variable amino acid means any amino acid. In some embodiments, one or more amino acid residues from the N-terminal fragment are duplicated in the C-terminal fragment. For example, in some embodiments, a methionine residue, followed by one or more variable amino acid residues, followed by one or more N-terminal amino acids are added to the N-terminal region of the C-terminal fragment.

In certain embodiments, the C-terminal, sigma-like fragment corresponds to M-X-601:883, wherein M=methionine; X is a variable amino acid; and 601-883 corresponds to the remainder of the C-terminal fragment of the polymerase, with residue 601 being repeated from the N-terminal fragment (SEQ ID NO:11). In some embodiments, the variable amino acid (X) is a lysine (K), and the sigma-like fragment corresponds to M-K-601:883 (SEQ ID NO:12).

Aspects of the invention relate to interaction between the N-terminal core fragment and the C-terminal sigma-like fragment. In some embodiments, association of the core fragment and the sigma-like fragment is increased by fusing each of the core fragment and sigma-like fragments to heterospecific protein interaction domains (PIDs). As used herein, a PID refers to any domain of a protein or peptide that mediates interaction with another protein or peptide. Thus, in some aspects, the association or interaction between the PIDs promotes or strengthens the formation of a complex comprising a core fragment and a sigma-like fragment.

In some embodiments, the core fragment and sigma-like fragments are each fused to PIDs that form coiled-coil interactions. In some aspects, the coiled coil is a structural motif that comprises two to five α-helices in parallel or antiparallel orientation. In some embodiments, two complimentary α-helices comprise the coiled coil motif. Typically, the N and C termini of the helices are easily accessible, facilitating linkage to other proteins, e.g., core fragment and sigma-like fragments. The most commonly observed type of coiled coil is left-handed, e.g., where each helix has a periodicity of seven (a heptad repeat), with anywhere from two to 200 of these repeats in a protein. This repeat is often denoted (a-b-c-d-e-f-g)ₙ in one helix, and (a′-b′-c′-d′-e′-f′-g′)ₙ in the complimentary helix. In this example, (a) and (d) are typically nonpolar, hydrophobic core residues (e.g., leucine, valine, isoleucine, etc.) found at the interface of the two helices, whereas (e) and (g) are solvent exposed polar residues (e.g., glutamate, lysine, etc.) that give specificity between the two helices through electrostatic interactions. Thus, for example, a system of the present disclosure may comprise a core fragment fused to one or more helices comprising (a-b-c-d-e-f-g)ₙ, while sigma-like fragments are fused to one or more helices comprising (a′-b′-c′-d′-e′-f-g′)ₙ. In such a system, the core fragment and sigma-like fragments would form a complex as a result of the interactions between (a-b-c-d-e-f-g)ₙ and (a′-b′-c′-d′-e′-f-g′)ₙ.

Coiled coil protein interacting domains are known in the art, and may be designed or identified using any available computational program. Several non-limiting embodiments of computational programs include SOCKET (e.g., as described in and incorporated by reference from Walshaw & Woolfson, J. Mol. Biol., 2001; 307(5), 1427-1450, available at the website of the Woolfson Group at the University of Bristol), COILS (e.g., as described in and incorporated by reference from Lupas et al., Science. 1991; 252:1162-1164, available at the ch.EMBnet.org website), PAIRCOIL (e.g., as described in and incorporated by reference from Berger et al., Proc Natl. Acad. Sci. USA. 1995; 92, 8259-8263, available at the groups.csail.mit.edu/cb/paircoil/cgi-bin/paircoil.cgi website, and MULTICOIL (e.g., as described by Wolf et al., Protein Sci. 1997; 6:1179-1189, available at the groups.csail.mit.edu/cb/multicoil/cgi-bin/multicoil.cgi website.

In some embodiments, the PIDs which form coiled coils are any of those disclosed in Table I of Müller et al., Methods Enzymol. 2000; 328, 261-282, incorporated herein by reference in its entirety. For example, PIDs which form coiled coils include, but are not limited to, leucine zippers (e.g., as found in the proteins GCN4, Fos, Jun, C/EBP, and variants or mutants thereof), peptide ‘velcro’ (e.g., as described by O'Shea et al., Curr Biol. 1993; 3(10):658-67), E-coil/K-coil (e.g., as described by Tripet et al., Protein Eng. 1996; 9, 1029), and WinZip-A2 and WinZip-B1 (e.g., as described by Arndt et al., Structure. 2002; (9):1235-48).

In some embodiments, the PIDs which form coiled coils are heterospecific synthetic coiled coil peptides called synzips, for example synzips 1-22. Detailed information on synzips 1-22 is disclosed in and incorporated by reference from SYNZIP specification sheets, available at the Keating lab web server at MIT. Synzips are also described in and incorporated by reference from Thompson et al., ACS Synth Biol. 2012; (4): 118-129. In some embodiments, the PIDs fused to either a core fragment or sigma-like fragments are synzip 17 (NEKEELKSKKAELRNRIEQLKQKREQLKQKIANLRKEIEAYK, SEQ ID NO:9) and/or synzip 18 (SIAATLENDLARLENENARLEKDIANLERDLAKLEREEAYF, SEQ ID NO:10). The sequence of the core fragment when the split occurs at reside 601 of T7 RNA polymerase, including the addition of a Gly-Ser linker and synzip 17 to the C-terminus, is provided by SEQ ID NO:4. A representative sequence of a T7 sigma-like fragment when the split occurs at reside 601, including the addition of synzip 18 and a Gly-Ser linker, is provided by SEQ ID NO:6. A representative sequence of a T3 sigma-like fragment when the split occurs at reside 601, including the addition of synzip 18, is provided by SEQ ID NO:7. A representative sequence of a K1FR sigma-like fragment when the split occurs at reside 601, including the addition of synzip 18, is provided by SEQ ID NO:8.

In some embodiments, the PIDs contemplated by the present disclosure include any of those disclosed on the website of Dr. Tony Pawson at Mount Sinai Hospital, Toronto. For example, PIDs include, but are not limited to, 14-3-3 domains, ADF domains, ANK repeats, ARM repeats, the BAR domain of amphiphysin, the BEACH domain, Bc1-2 homology (BH) domains (e.g., BH1, BH2, BH3, BH4), BIR domains, BRCT domains, bromodomains, BTB/POZ domains, C1 domains, C2 domains, caspase recruitment domains (CARDs), clathrin assembly lymphoid myeloid (CALM) domains, calponin homology (CH) domains, chromatin organization modifier (CHROMO/Chr) domains, CUE domains, death (DD) domains, death-effector (DED) domains, DEP domains, Dbl homology (DH) domains, EF-hand (EFh) domains, Eps15 homology (EH) domains, epsin NH2-terminal homology (ENTH) domains, Ena/Vasp Homology domain 1 (EVH1 domains), F-box domains, FERM domains, FF domains, formin homology-2 (FH2) domains, Forkhead-Associated (FH) domains, FYVE (Fab-1, YGL023, Vps27, and EEA1) domains, GAT (GGA and Toml) domains, gelsolin/severin/villin homology (GEL) domains, GLUE (from GRAM-like ubiquitin-binding in EAP45) domains, GRAM (from glucosyltransferases, Rab-like GTPase activators and myotubularins) domains, GRIP domains, glycine-tyrosine-phenylalanine (GYF) domains, HEAT (from Huntington, Elongation Factor 3, PR65/A, TOR) domains, HECT (from Homologous to the E6-AP Carboxyl Terminus) domains, IQ domains, LIM domains, leucine-rich repeat (LRR) domains, malignant brain tumor (MBT) domains, Mad homology 1 (MH1) domains, MH2 domains, MIU (from Motif Interacting with Ubiquitin) domains, NZF (Np14 zinc finger) domains, PAS (Per-ARNT-Sim) domains, Phox and Beml (PB1) domains, PDZ (from postsynaptic density 95, PSD-85; discs large, Dlg; zonula occludens-1, ZO-1) domains, Pleckstrin-homology (PH) domains, Polo-Box domains, phosphotyrosine binding (PTB) domains, pumilio (Puf) domains, PWWP domains, Phox homology (PX) domains, RGS (Regulator of G protein Signaling) domains, RING finger domains, SAM (Sterile Alpha Motif) domains, shadow chromo (CSD or SC) domains, Src-homology 2 (SH2) domains, Src-homology 3 (SH3) domains, SOCS (from suppressors of cytokine signaling) box domains, SPRY domains, START (from steroidogenic acute regulatory protein (StAR) related lipid transfer) domains, SWIRM domains, Toll/Il-1 Receptor (TIR) domains, tetratricopeptide repeat (TPR) motif domains, TRAF domains, SNARE (from soluble NSF attachment protein (SNAP) receptors) domains (e.g., T-SNARE), Tubby domains, tudor domains, ubiquitin-associated (UBA) domains, UEV (Ubiquitin E2 variant) domains, ubiquitin-interacting motif (UIM) domains, beta-domains of the von Hippel-Lindau tumor suppressor protein (VHLβ), VHS (from Vps27p, Hrs and STAM) domains, WD40 repeat domains, and WW domains.

PIDs can be linked to the core fragment and/or sigma-like fragment with or without a linker. It should be appreciated that any PIDs and any linkers can be compatible with aspects of the invention. In some embodiments, the linker is flexible. The linker can be composed of amino acids. In some embodiments, the linker is composed of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more than 50 amino acids. In some embodiments, the linker is 5-7 amino acids. In some embodiments, the linker is a Gly-Ser linker.

The sequences of the core fragments and/or the sigma-like fragments can be engineered. As used herein, engineering of a core fragment or sigma-like fragment refers to changing at least one nucleotide within the core fragment or sigma-like fragment relative to the sequence prior to it being engineered. Engineering of a sigma-like fragment can lead to its having different promoter DNA sequence specificity than it had prior to engineering, thereby increasing the repertoire of DNA binding specificities conferred by a collection of sigma-like fragments on a core fragment. In some embodiments, the sigma-like fragment is engineered within the recognition loop portion of the sigma-like fragment. Systems associated with aspects of the invention can also include nucleic acids comprising promoter DNA sequences that are bound by sigma-like factors. Promoter sequences can be engineered to change their sigma-like factor binding specificity. In some embodiments, the T7 RNA polymerase is engineered to have a non-native promoter DNA sequence specificity.

Aspects of the invention encompass sigma-like fragment-promoter interactions that are orthogonal. As used herein, an orthogonal sigma-like fragment-promoter interaction refers to an interaction that does not exhibit “cross-talk,” meaning that the sigma-like fragment does not interfere with or regulate transcriptional regulatory elements other than the transcriptional regulatory elements containing the cognate promoter of the sigma-like fragment. In some embodiments, a promoter is activated at least 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold or more than 20-fold more by its cognate sigma-like fragment than by any non-cognate sigma-like fragment.

The promoter DNA sequences can be operably linked to a reporter sequence and/or a protein-coding sequence. As used herein, a coding sequence and regulatory sequences are said to be “operably” joined or linked when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide. It should be appreciated that any reporter sequence and/or protein coding sequence can be compatible with aspects of the invention and can be operably linked or joined to a promoter sequence.

The core fragment and sigma-like fragments can be independently expressed, meaning that the expression of the core fragment is under separate regulatory control than the expression of the sigma-like fragments. The core fragment and/or the sigma-like fragment can in some embodiments be expressed constitutively. In some embodiments, the core fragment and/or sigma-like fragment are expressed under the control of inducible promoters. Expression of the core fragment and/or sigma-like fragment can be from a low, medium or high copy number plasmid. In some embodiments, the core fragment is expressed from a low copy number or single copy plasmid, while each sigma-like fragment is expressed from a medium-copy number plasmid. In some embodiments, the core fragment is expressed constitutively while the sigma-like fragments are expressed under the control of inducible promoters. In some embodiments, the expression of the sigma-like fragments is regulated by inputs to the system, such as conditions that the system is exposed to.

Aspects of the invention relate to recombinant expression of proteins and protein fragments. As used herein “recombinant” and “heterologous” are used interchangeably to refer to a relationship between a cell and a polynucleotide wherein the polynucleotide originates from a foreign species, or, if from the same species, is modified from its original (native) form. Further aspects of the invention relate to the use of recombinant proteins and protein fragments in genetic circuits.

As used herein, a genetic circuit refers to a collection of recombinant genetic components that responds to one or more inputs and performs a specific function, such as the regulation of the expression of one or more genetic components and/or regulation of an ultimate output of the circuit. In some embodiments, genetic circuit components can be used to implement a Boolean operation in living cells based on an input detected by the circuit.

Aspects of the invention relate to recombinant cells that comprise logic functions that influence how each cell responds to one more input signals. In some embodiments, a logic function can be a logic gate. As used herein, a “logic function,” “logic gate” or “logic operation” refers to a fundamental building block of a circuit. Several non-limiting examples of logic gates compatible with aspects of the invention include AND, OR, NOT (also called INVERTER), NAND, NOR, IDENTITY, XOR, XNOR, EQUALS, IMPLIES, ANDN and N-IMPLIES gates. The use of Logic Gates is known to those of skill in the art (see, e.g. Horowitz and Hill (1990) The Art of Electronics, Cambridge University Press, Cambridge). Genetic circuits can comprise any number of logic gates. In some embodiments, NOR gates can comprise a transcriptional repressors and a transcriptional repressor target DNA sequence, while AND gates can comprise a transcriptional activator and a transcriptional activator target DNA sequence.

Genetic circuits can be comprised of one or more logic gates that process one or more input signals and generate an output according to a logic design. In some embodiments, genetic components respond to biological inputs and are regulated using combinations of repressors and activators. Non-limiting examples of logic gates using genetic components have been described (Tamsir et al. (2011) Nature 469(7329):212-215). In some embodiments, the genetic circuit functions as, for example, a switch, oscillator, pulse generator, latch, flip-flop, feedforward loop, or feedback loop.

Genetic circuits can comprise other components such as other transcriptional activators and transcriptional repressors. Non-limiting examples of transcriptional activators and transcriptional repressors are disclosed in and incorporated by reference from WO 2012/170436 (see, e.g., pages 27-40; Table 1 on pages 28-30; and Tables 2 and 3 on pages 36-38, of WO 2012/170436).

Aspects of the invention relate to recombinant host cells that express regulatory components and/or genetic circuits. It should be appreciated that the invention encompasses any type of recombinant cell, including prokaryotic and eukaryotic cells. As used herein, a “host cell” refers to a cell that is capable of replicating and/or transcribing and/or translating a recombinant gene. A host cell can be a prokaryotic cell or a eukaryotic cell and can be in vitro or in vivo. In some embodiments, a host cell is within a transgenic animal or plant.

In some embodiments the recombinant cell is a bacterial cell, such as Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp. and Pantoea spp. The bacterial cell can be a Gram-negative cell such as an Escherichia coli (E. coli) cell, or a Gram-positive cell such as a species of Bacillus.

In other embodiments, the cell is an algal cell, a plant cell, an insect cell or a mammalian cell. In certain embodiments, the mammalian cell is a human cell.

In some embodiments, multicellular systems described herein contain cells that originate from more than one different type of organism.

Aspects of the invention relate to recombinant expression of one or more genes encoding components of genetic circuits. It should be appreciated that some cells compatible with the invention may express an endogenous copy of one or more of the genes associated with the invention as well as a recombinant copy. In some embodiments, if a cell has an endogenous copy of one or more of the genes associated with the invention, then the methods will not necessarily require adding a recombinant copy of the gene(s) that are endogenously expressed.

According to aspects of the invention, cell(s) that recombinantly express one or more components of genetic circuits are provided. It should be appreciated that the genes associated with the invention can be obtained from a variety of sources. As one of ordinary skill in the art would be aware, homologous genes for any of the genes described herein could be obtained from other species and could be identified by homology searches, for example through a protein BLAST search, available at the National Center for Biotechnology Information (NCBI) internet site (ncbi.nlm.nih.gov). Genes associated with the invention can be PCR amplified from DNA from any source of DNA which contains the given gene. In some embodiments, genes associated with the invention are synthetic. Any means of obtaining a gene associated with the invention are compatible with the instant invention. Aspects of the invention encompass any cell that recombinantly expresses one or more components of a genetic circuit as described herein.

One or more of the genes associated with the invention can be expressed in a recombinant expression vector. As used herein, a “vector” may be any of a number of nucleic acids into which a desired sequence or sequences may be inserted, such as by restriction and ligation, for transport between different genetic environments or for expression in a host cell. Vectors are typically composed of DNA, although RNA vectors are also available. Vectors include, but are not limited to: plasmids, fosmids, phagemids, virus genomes and artificial chromosomes.

A cloning vector is one which is able to replicate autonomously or integrated in the genome in a host cell, and which can be further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host cell such as a host bacterium or just a single time per host before the host reproduces by mitosis. In the case of phage, replication may occur actively during a lytic phase or passively during a lysogenic phase.

An expression vector is one into which a desired DNA sequence may be inserted, for example by restriction and ligation, such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

When the nucleic acid molecule that encodes any of the genes associated with the claimed invention is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Fourth Edition, Cold Spring Harbor Laboratory Press, 2012. Cells are genetically engineered by the introduction into the cells of heterologous DNA (RNA). That heterologous DNA (RNA) is placed under operable control of transcriptional elements to permit the expression of the heterologous DNA in the host cell. A nucleic acid molecule that comprises a gene associated with the invention can be introduced into a cell or cells using methods and techniques that are standard in the art.

In some embodiments, it may be advantageous to use a cell that has been optimized for expression of one or more polypeptides. As used herein, “optimizing expression” of a polypeptide refers to altering the nucleotide sequences of a coding sequence for a polypeptide to alter the expression of the polypeptide (e.g., by altering transcription of an RNA encoding the polypeptide) to achieve a desired result. In some embodiments, the desired result can be optimal expression, but in other embodiments the desired result can be simply obtaining sufficient expression in a heterologous host cell to test activity (e.g., DNA sequence binding) of the polypeptide.

In other embodiments, optimizing can also include altering the nucleotide sequence of the gene to alter or eliminate native transcriptional regulatory sequences in the gene, thereby eliminating possible regulation of expression of the gene in the heterologous host cell by the native transcriptional regulatory sequence(s). Optimization can include replacement of codons in the gene with other codons encoding the same amino acid. The replacement codons can be those that result in optimized codon usage for the host cell, or can be random codons encoding the same amino acid, but not necessarily selected for the most “preferred” codon in a particular host cell.

In some embodiments, it may be optimal to mutate the cell prior to or after introduction of recombinant gene products. In some embodiments, screening for mutations that lead to enhanced or reduced production of one or more genes may be conducted through a random mutagenesis screen, or through screening of known mutations. In some embodiments, shotgun cloning of genomic fragments can be used to identify genomic regions that lead to an increase or decrease in production of one or more genes, through screening cells or organisms that have these fragments for increased or decreased production of one or more genes. In some instances, one or more mutations may be combined in the same cell or organism. Recombinant gene expression can involve in some embodiments expressing a gene on a plasmid and/or integrating the gene into the chromosomal DNA of the cell. For example, nucleic acid molecules can be introduced by standard protocols such as transformation including chemical transformation and electroporation, transduction, particle bombardment, etc. Expressing the nucleic acid molecule can also be accomplished by integrating the nucleic acid molecule into the genome.

Optimization of protein expression may also require in some embodiments that a gene be modified before being introduced into a cell such as through codon optimization for expression in a bacterial cell. Codon usages for a variety of organisms can be accessed in the Codon Usage Database (http://www.kazusa.or.jp/codon/).

Protein engineering can also be used to optimize expression or activity of a protein. In certain embodiments a protein engineering approach could include determining the three dimensional (3D) structure of a protein or constructing a 3D homology model for the protein based on the structure of a related protein. Based on 3D models, mutations in a protein can be constructed and incorporated into a cell or organism, which could then be screened for increased or decreased production of a protein or for a given feature or phenotype.

A nucleic acid, polypeptide or fragment thereof described herein can be synthetic. As used herein, the term “synthetic” means artificially prepared. A synthetic nucleic acid or polypeptide is a nucleic acid or polypeptide that is synthesized and is not a naturally produced nucleic acid or polypeptide molecule (e.g., not produced in an animal or organism). It will be understood that the sequence of a natural nucleic acid or polypeptide (e.g., an endogenous nucleic acid or polypeptide) may be identical to the sequence of a synthetic nucleic acid or polypeptide, but the latter will have been prepared using at least one synthetic step.

Aspects of the invention thus involve recombinant expression of genes encoding RNA polymerases, functional modifications and variants of the foregoing, as well as uses relating thereto. Homologs and alleles of the nucleic acids associated with the invention can be identified by conventional techniques. Also encompassed by the invention are nucleic acids that hybridize under stringent conditions to the nucleic acids described herein. The term “stringent conditions” as used herein refers to parameters with which the art is familiar. Nucleic acid hybridization parameters may be found in references which compile such methods, e.g. Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York. More specifically, stringent conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄(pH7), 0.5% SDS, 2 mM EDTA). SSC is 0.15M sodium chloride/0.015M sodium citrate, pH 7; SDS is sodium dodecyl sulphate; and EDTA is ethylenediaminetetracetic acid. After hybridization, the membrane upon which the DNA is transferred is washed, for example, in 2×SSC at room temperature and then at 0.1-0.5×SSC/0.1×SDS at temperatures up to 68° C.

There are other conditions, reagents, and so forth which can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of nucleic acids of the invention (e.g., by using lower stringency conditions). The skilled artisan also is familiar with the methodology for screening cells and libraries for expression of such molecules which then are routinely isolated, followed by isolation of the pertinent nucleic acid molecule and sequencing.

In general, homologs and alleles typically will share at least 75% nucleotide identity and/or at least 90% amino acid identity to the sequences of nucleic acids and polypeptides, respectively, in some instances will share at least 90% nucleotide identity and/or at least 95% amino acid identity and in still other instances will share at least 95% nucleotide identity and/or at least 99% amino acid identity. In some embodiments, homologs and alleles share at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or more than 99% nucleotide identity and/or at least 90%, 91%, 92%, 93%, 94%, 95%, 96%, 97%, 98%, 99% or more than 99% amino acid identity. The homology can be calculated using various, publicly available software tools developed by NCBI (Bethesda, Md.) that can be obtained through the NCBI internet site. Exemplary tools include the BLAST software, also available at the NCBI internet site (www.ncbi.nlm.nih.gov). Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are embraced by the invention.

The invention also includes degenerate nucleic acids which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code. The invention also embraces codon optimization to suit optimal codon usage of a host cell.

The invention also provides modified nucleic acid molecules which include additions, substitutions and deletions of one or more nucleotides. In preferred embodiments, these modified nucleic acid molecules and/or the polypeptides they encode retain at least one activity or function of the unmodified nucleic acid molecule and/or the polypeptides, such as enzymatic activity. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules which encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules which encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

The invention embraces variants of polypeptides. As used herein, a “variant” of a polypeptide is a polypeptide which contains one or more modifications to the primary amino acid sequence of the polypeptide. Modifications which create a variant can be made to a polypeptide 1) to reduce or eliminate an activity of a polypeptide; 2) to enhance a property of a polypeptide; 3) to provide a novel activity or property to a polypeptide, such as addition of an antigenic epitope or addition of a detectable moiety; or 4) to provide equivalent or better binding between molecules (e.g., an enzymatic substrate). Modifications to a polypeptide are typically made to the nucleic acid which encodes the polypeptide, and can include deletions, point mutations, truncations, amino acid substitutions and additions of amino acids or non-amino acid moieties. Alternatively, modifications can be made directly to the polypeptide, such as by cleavage, addition of a linker molecule, addition of a detectable moiety, such as biotin, addition of a fatty acid, and the like. Modifications also embrace fusion proteins comprising all or part of the amino acid sequence. One of skill in the art will be familiar with methods for predicting the effect on protein conformation of a change in protein sequence, and can thus “design” a variant of a polypeptide according to known methods. One example of such a method is described by Dahiyat and Mayo in Science 278:82-87, 1997, whereby proteins can be designed de novo. The method can be applied to a known protein to vary a only a portion of the polypeptide sequence. By applying the computational methods of Dahiyat and Mayo, specific variants of a polypeptide can be proposed and tested to determine whether the variant retains a desired conformation.

In general, variants include polypeptides which are modified specifically to alter a feature of the polypeptide unrelated to its desired physiological activity. For example, cysteine residues can be substituted or deleted to prevent unwanted disulfide linkages. Similarly, certain amino acids can be changed to enhance expression of a polypeptide by eliminating proteolysis by proteases in an expression system (e.g., dibasic amino acid residues in yeast expression systems in which KEX2 protease activity is present).

Mutations of a nucleic acid which encode a polypeptide preferably preserve the amino acid reading frame of the coding sequence, and preferably do not create regions in the nucleic acid which are likely to hybridize to form secondary structures, such a hairpins or loops, which can be deleterious to expression of the variant polypeptide.

Mutations can be made by selecting an amino acid substitution, or by random mutagenesis of a selected site in a nucleic acid which encodes the polypeptide. Variant polypeptides are then expressed and tested for one or more activities to determine which mutation provides a variant polypeptide with the desired properties. Further mutations can be made to variants (or to non-variant polypeptides) which are silent as to the amino acid sequence of the polypeptide, but which provide preferred codons for translation in a particular host. The preferred codons for translation of a nucleic acid in, e.g., E. coli, are well known to those of ordinary skill in the art. Still other mutations can be made to the noncoding sequences of a gene or cDNA clone to enhance expression of the polypeptide. The activity of variant polypeptides can be tested by cloning the gene encoding the variant polypeptide into a bacterial or mammalian expression vector, introducing the vector into an appropriate host cell, expressing the variant polypeptide, and testing for a functional capability of the polypeptides as disclosed herein.

The skilled artisan will also realize that conservative amino acid substitutions may be made in polypeptides to provide functionally equivalent variants of the foregoing polypeptides, i.e., the variants retain the functional capabilities of the polypeptides. As used herein, a “conservative amino acid substitution” refers to an amino acid substitution which does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York. Exemplary functionally equivalent variants of polypeptides include conservative amino acid substitutions in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In general, it is preferred that fewer than all of the amino acids are changed when preparing variant polypeptides. Where particular amino acid residues are known to confer function, such amino acids will not be replaced, or alternatively, will be replaced by conservative amino acid substitutions. Preferably, 1, 2, 3, 4, 5, 6, 7, 8, 9, 10, 11, 12, 13, 14, 15, 16, 17, 18, 19, 20 residues can be changed when preparing variant polypeptides. It is generally preferred that the fewest number of substitutions is made. Thus, one method for generating variant polypeptides is to substitute all other amino acids for a particular single amino acid, then assay activity of the variant, then repeat the process with one or more of the polypeptides having the best activity.

Conservative amino-acid substitutions in the amino acid sequence of a polypeptide to produce functionally equivalent variants of the polypeptide typically are made by alteration of a nucleic acid encoding the polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a polypeptide.

Genetic circuits described herein can contain a variety of transcriptional regulatory elements. As used herein, a “transcriptional regulatory element” refer to any nucleotide sequence that influences transcription initiation and rate, or stability and/or mobility of a transcript product. Regulatory sequences include, but are not limited to, promoters, promoter control elements, protein binding sequences, 5′ and 3′ UTRs, transcriptional start sites, termination sequences, polyadenylation sequences, introns, etc. Such transcriptional regulatory sequences can be located either 5′-, 3′-, or within the coding region of the gene and can be either promote (positive regulatory element) or repress (negative regulatory element) gene transcription.

Aspects of the invention encompass a non-transitory computer readable storage medium encoded with instructions, executable by a processor, for designing a host cell and a computer product comprising a computer readable medium encoded with a plurality of instructions for controlling a computing system to perform an operation for designing a host cell. As used herein, “computer-readable medium” refers to any media that is involved in providing one or instructions to a processor for execution. Computer-readable media can be anything that a computer is able to read, such as, for example, disks, magnetic tape, CD-ROMs, any other optical medium, punch cards, paper tape, any other physical medium with patterns of holes, a RAM, PROM, and EPROM, a FLASH-EPROM, any other memory chip or cartridge or a carrier wave.

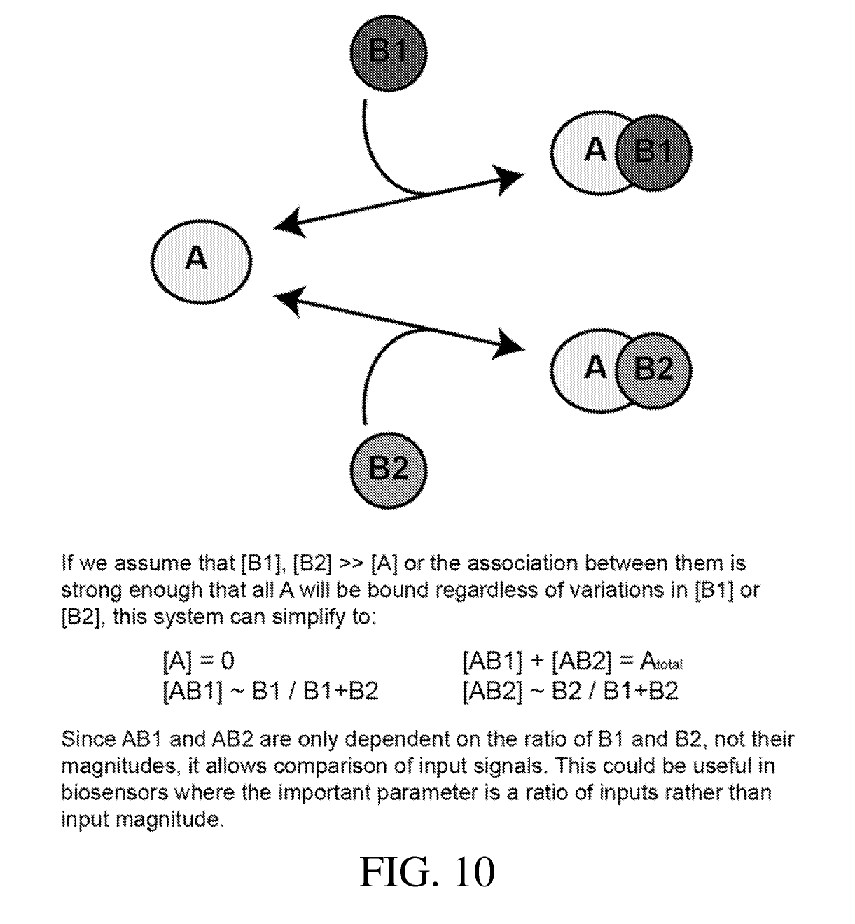

In some embodiments, systems described herein are used in methods for controlling RNA transcription of one or more DNA sequences by placing the one or more DNA sequences under the transcriptional control of the system. The one or more DNA sequences can be operably linked to a promoter sequence that is specifically bound by a protein complex comprising a sigma-like fragment and a core fragment of a system or bisected polymerase protein described herein. In some embodiments, the ratio of the expression of the set of sigma-like fragments determines the output of the system (FIG. 10).

Aspects of the invention relate to a novel regulatory system that has not been previously built or attempted. While it follows similar design principles as natural systems, those natural systems are not fully accessible for genetic engineering because they tie into key aspects of cells so perturbations can be quite deleterious. This regulatory system opens up new ways to regulate genetic circuits, either by implementing a type of “trade-off” logic, where expression of one pathway decreases as another increases, or by implementing a ratio calculator that allows the measurement of the ratio between two input signals and returns a single protein expression level as a result.

In some aspects, the system is designed and controlled to avoid or reduce toxicity that can accompany strong RNA polymerases. For example, the conserved protein can be expressed at a level below that which results in toxicity in a cell. The variable, sigma-like proteins can be expressed only when expression is wanted or induced by one or more specific events or conditions, such as based on one or more inputs from a genetic circuit.

Aspects of the novel regulatory system described herein can be used to build complex genetic regulatory systems. The logic provided by this system is unique from the current toolbox of synthetic biology parts and provides utility in systems level engineering. Furthermore the protein building blocks (T7 RNA polymerases) are widely used in industry in a number of processes, so aspects of the invention can be used with many existing systems.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference, including the entire contents of WO 2012/170436 and International Patent Application No.: PCT/US2013/032145.

### EXAMPLES

### Example 1

### Identification of Split Sites in RNA Polymerase

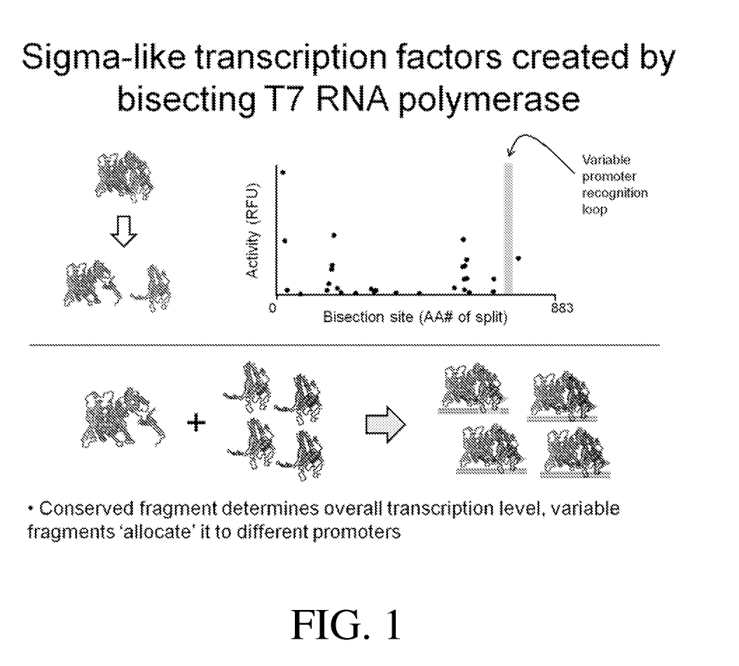

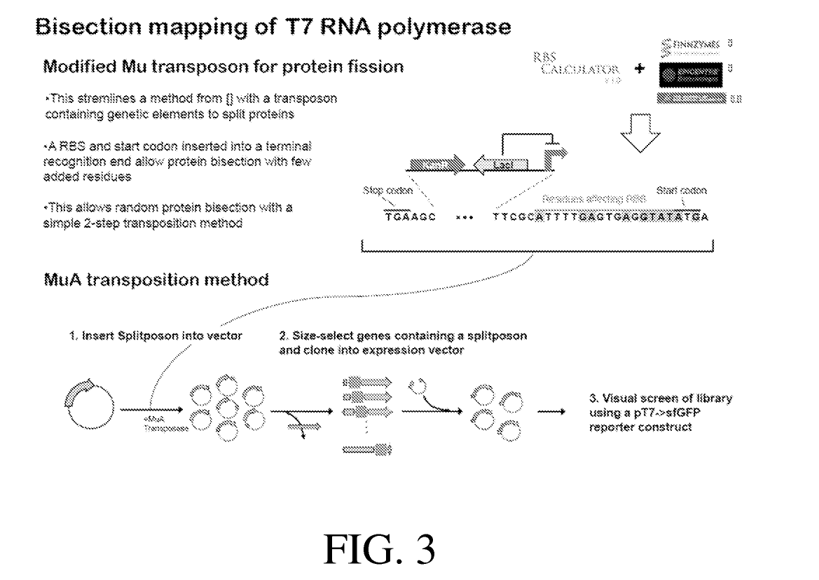

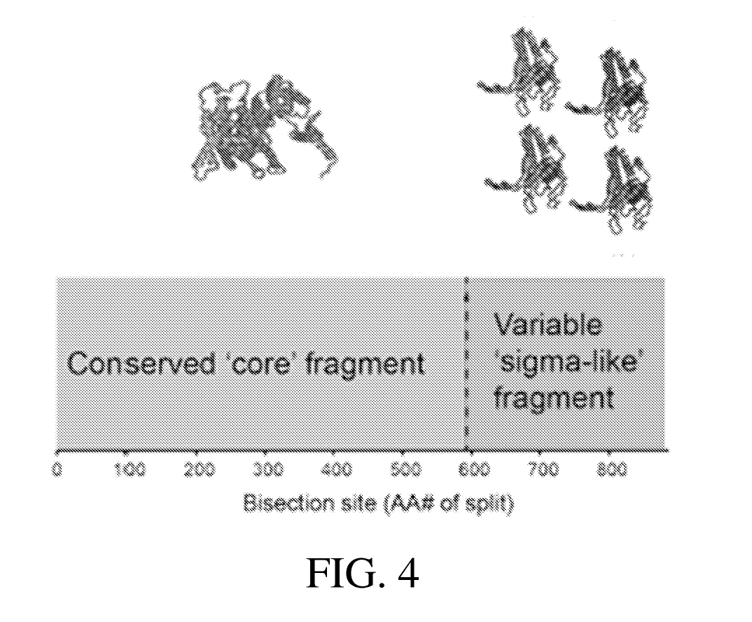

An RNA polymerase protein (T7 RNA polymerase) was split at a library of random sites using Mu transposon (“splitposon”) as shown in FIG. 3 to produce a set of two proteins (a conserved or core fragment and a variable or sigma-like fragment; FIG. 4). Locations were identified at which the two fragments of the protein retain function (FIGS. 1-3). Analysis of the proteins in the library determined several locations at which highly functional split sites clustered, indicating that the protein can tolerate being split at several different regions.

In a non-limiting embodiment depicted in FIG. 1, applying functional split sites to a set of four orthogonal T7 RNA polymerases, which have mutations in one region—indicated as the “Variable Specificity Loop Region” or “Variable Promoter Recognition Loop Region”—yielded one conserved core fragment and four variable sigma-like fragments (FIG. 1). For example, a split at amino acid 593 was applied to a library of orthogonal T7 RNA polymerases, which vary mainly in a variable specificity loop region from amino acids 739-767. Hence, the split divided them into a conserved core fragment that does not vary between polymerases, and a variable sigma-like fragment that does vary between polymerases. Since the variable sigma-like fragments re-fold with the conserved core fragment and target it to orthogonal sites, the variable sigma-like factors are analogous to sigma factors, and the conserved core fragment is analogous to the ‘core’ polymerase.

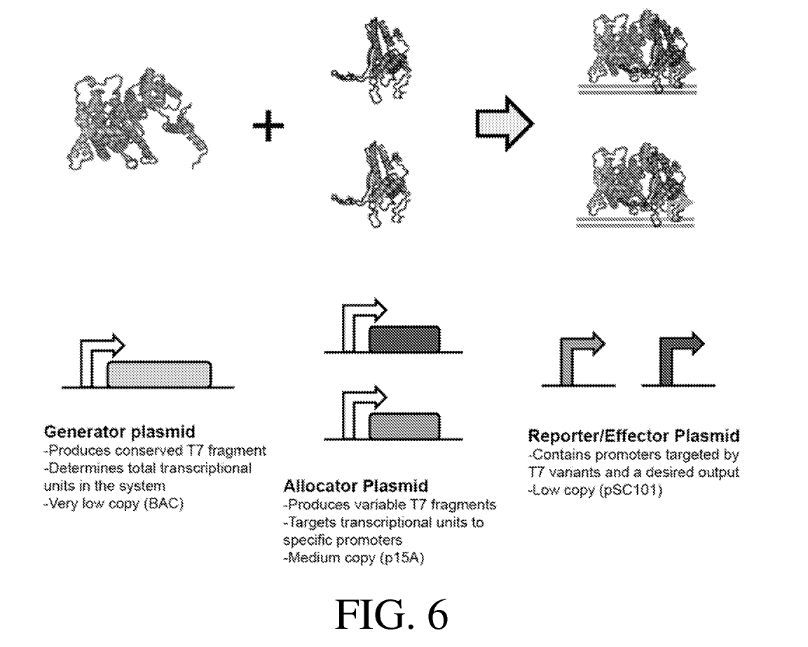

These fragments were further tested on a system of three plasmids (FIG. 6) to demonstrate that the conserved fragment and variable fragments can be expressed in trans and that the specificity of the split polymerase is dictated by the variable region. In some embodiments, the core T7 fragment was a conserved fragment of T7 polymerase produced by a generator plasmid. Variable fragments (K1F or T3, see Example 3 for details) were produced by allocator plasmids. The reporter plasmids contained a promoter recognized by either K1F or T3 driving expression of a superfolder GFP (sfGFP) protein. This test demonstrated that orthogonal targeting of the core fragment of T7 to different promoters was achieved by different variable fragments.

In some embodiments, two different promoters (pJ23101 or pJ23105) were used to drive different levels of expression of the core T7 fragment in generator plasmids. The variable fragment (T3) was produced by an allocator plasmid. The reporter plasmid contained a promoter recognized by T3 driving expression of a superfolder GFP (sfGFP) protein. This test demonstrated that the level of the core fragment influences the transcriptional activity of the system.

### Example 2

### Bisection Mapping of T7 RNA Polymerase

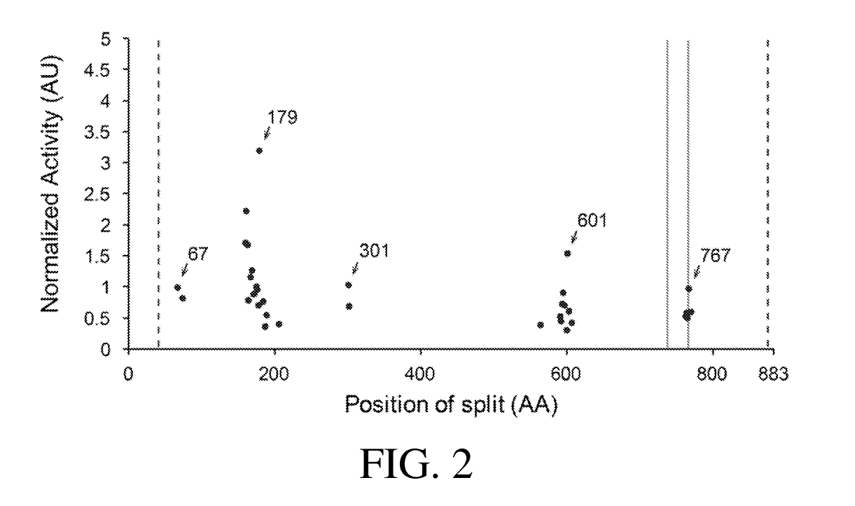

Thorough second round split T7 RNA polymerase mapping was used to identify further split sites. An MuA transposon was designed to contain stop codons on one end, and an inducible promoter+start codon on the other end. This transposon was randomly inserted into a region of T7 RNA polymerase to generate a library, and then the library was transformed into cells with a T7 RNA polymerase dependent promoter driving a fluorescent protein (mrfp) to assay for activity. This library was initially screened on plates to find 384 very active clones. These were then measured in liquid culture to find the most active 192. These 192 were assayed in detail and sequenced to map the split points, the results of which are shown in FIG. 2. From those 192 clones, 36 unique in-frame split points were found, along with 19 out-of-frame split points. (The out-of-frame split points were expressed from pre-existing start codons in the polymerase sequence and were ignored in further analysis.) The split position is defined as the length of the N-terminal fragment. In some embodiments, due to the splitting method, the terminal amino acid is repeated on both fragments and a methionine plus one variable residue is added to the beginning of the C-terminal fragment. (Hence, the split site of 601 represents the fragments 1:601 and M-X-601:883).

Cells containing a plasmid from the split T7 library and a plasmid containing pT7->mrfp were inoculated into 0.5 mL 2YT+antibiotics and grown to saturation overnight. These overnight cultures were diluted 1:20 into 0.15 mL 2YT+antibiotics+10 μM IPTG (to induce expression of the T7 fragments) and grown for 6 hours at 37° C. 1000 rpm. The fluorescence was measured on a flow cytometer. The geometric mean fluorescence of each sample was calculated and normalized to the average of all of the measured values for the day. Data shown in FIG. 2 is the average of four independent inductions taken over four days. If more than one clone was found to be split at a given point, their activities were averaged.

Based on the information from this assay, five ‘seams’ or split sites were identified at which T7 RNA polymerase could be functionally bisected. These are located at approximately amino acids 67-74, 160-206, 301-302, 564-607, and 763-770, plus some surrounding sequence. From these five seams, the most active split variant was selected in each (marked on FIG. 2): amino acids 67, 179, 301, 601 and 767. These were re-built/re-assayed to confirm activity. Additionally, ‘synzip’ coiled-coil domains were added to increase the association of the split fragments (Thompson et al. (2012) SYNZIP Protein Interaction Toolbox: in Vitro and in Vivo Specifications of Heterospecific Coiled-Coil Interaction Domains, ACS Synth Biol 1(4):118-129). Synzip 17 and 18 were chosen because they bind to each other in an antiparallel fashion, with synzip 17 fused to the end of the N-terminal fragment and synzip 18 fused to the beginning of the C-terminal fragment. The synzips were attached to the T7 domains with flexible linker regions comprising 5-7 amino acids.

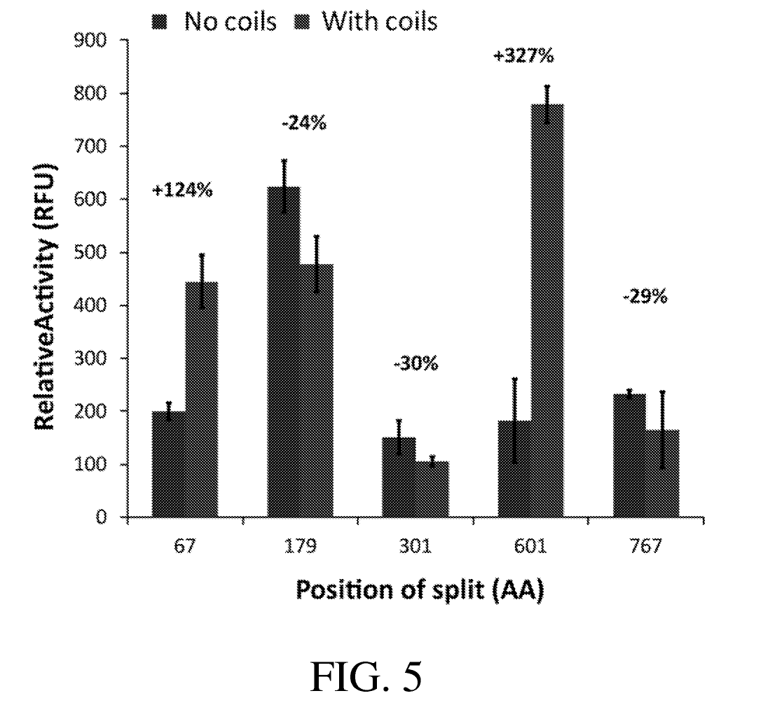

Fragments generated by splitting T7 RNA polymerase at points from each of the five seams indicated in FIG. 2 without (“no coils”) and with (“with coils”) synzip domains were assayed for function. Cells were produced that include plasmids expressing a set of a core fragment and a sigma-like fragment, and a T7 reporter plasmid producing a fluorescent protein. Growth of cells and testing of fluorescence was performed as described in Example 3. Data shown is from four technical replicates (FIG. 5). The numbers above each set of bars represent the percent increase or decrease in activity with synzip domains added as compared to the fragments without added synzip domains.

### Example 3

### Building a Sigma-Like Control System

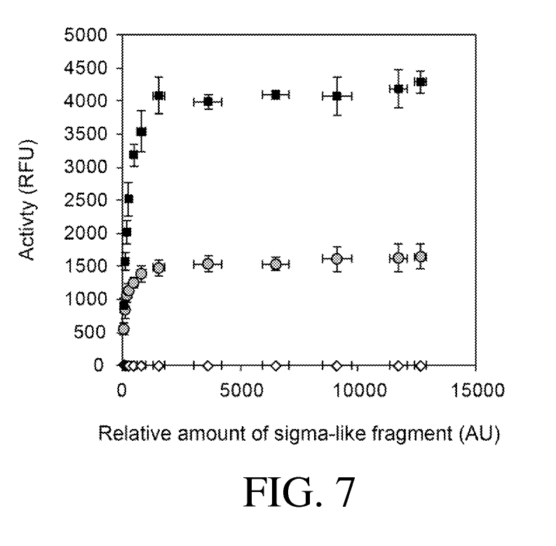

The T7 RNA polymerase was split at amino acid 601 plus synzips for building the sigma-like control system. Since the fragment from 601-883 contains the variable promoter recognition loop, it is referred to as the ‘sigma-like’ fragment, while the 1-601 fragment is referred to as ‘core.’ The system was reorganized such that the core fragment was expressed constitutively from a single copy plasmid, the variable sigma-like fragments were expressed from a medium-high copy plasmid, and the activity of T7 RNA polymerase was measured off of a fluorescent reporter on a low copy plasmid (FIG. 6). This system demonstrates that both fragments of the polymerase are necessary for function, and shows that the core fragment can be saturated with the sigma-like fragments (FIG. 7). As the sigma-like fragments' expression level is increased, activity goes up until it reaches a level where all of the core fragments are bound to sigma-like fragments. This level of activity is tied to the expression level of the core fragment.

Cells containing the three test plasmids (constitutive expression of core, pT7->sfgfp reporter, and pTac->sigma-like fragment) were inoculated into 0.5 mL LB+antibiotics and grown to saturation overnight. The overnight cultures were diluted 1:200 into 0.15 mL LB+antibiotics+IPTG and grown at 37° C., 1000 rpm for 6 hours. The fluorescence of the cells was quantified using a flow cytometer. The data shown represents three replicates performed on separate days.

After verifying the functionality of the split site+synzips, as well as the three plasmid testing system, a set of variable sigma-like fragments was created that recognize different promoters. This was done by swapping out the recognition loop portion of the T7 RNA polymerase, as described for full length T7 polymerase in Temme et al. (2012) Nucleic Acids Research 40(17):8773-8781. Three successful variants were engineered: T7 (wild-type plus a mutation that reduces its strength and toxicity somewhat) and T3, which are from Temme et al. and are incorporated by reference from (SYNTHETIC BIOLOGY TOOLS, U. S. Patent Publication No. 20130005590) and K1FR (K1F, described in Temme et al., was mutated to be more active in this system).

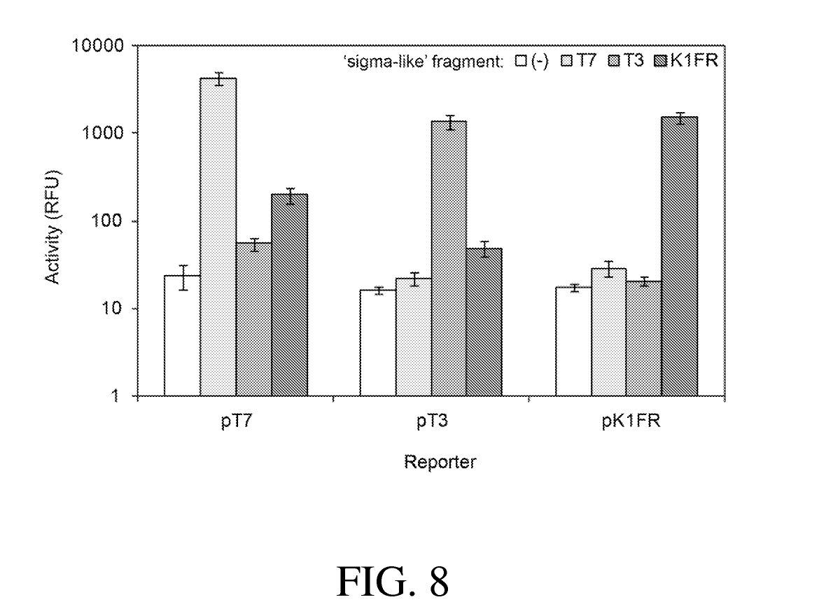

Cells containing the three test plasmids (constitutive expression of core, promoter->sfgfp reporter, and pTac->sigma-like fragment) were inoculated into 0.5 mL LB+antibiotics and grown to saturation overnight. The overnight cultures were diluted 1:200 into 0.15 mL LB+antibiotics+100 μM IPTG and grown at 37° C., 1000 rpm for 6 hours. The fluorescence of the cells was quantified using a flow cytometer (FIG. 8). The assay was performed on three separate days, with three technical replicates per day. Data shown is the average value over all three days, with error bars representing the standard deviation between the mean values on each day.

Two of the sigma-like fragments were tested together to assess the functionality of the system. The intention was to have the level of core polymerase fragments set the total amount of polymerase units accessible to the system and then have the sigma-like fragments ‘compete’ for the core fragments. In this system, the relative amounts of the sigma-like factors determine how much of the available core fragment each binds. For example, if sigma-like fragment 1 is three times as abundant as sigma-like fragment 2, it may be expected that fragment 1 binds 75% of the available core, while fragment 2 binds 25%. This ideal system is shown in a simplified form in FIG. 10. In order to test whether this type of interaction was possible, the medium strength plasmid was changed to express both the T3 sigma-like fragment from a pTac inducible promoter and the T7 sigma-like fragment from a pTet inducible promoter.

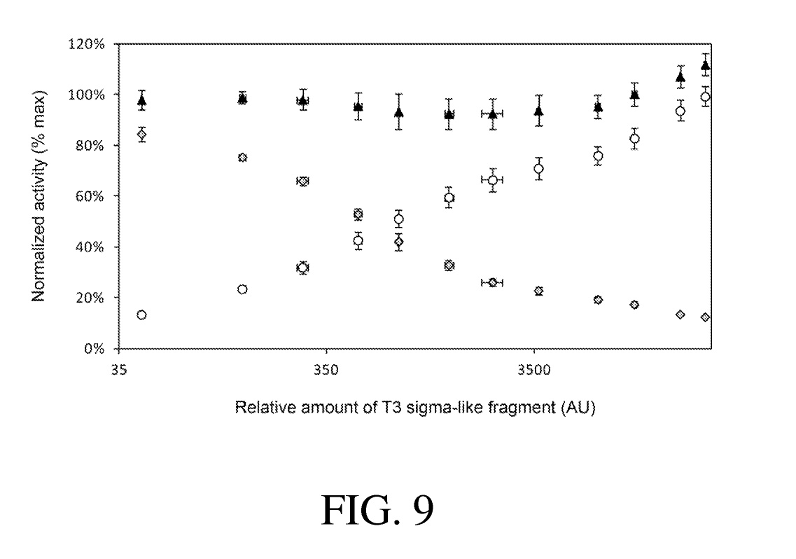

Four strains of cells were used. All contained a plasmid expressing a constant level of the core fragment. One contained a plasmid with pTac->sfgfp to measure the relative amount of the T3 sigma factor being expressed. The other three contained a plasmid expressing both the T7 fragment and the T3 fragment, along with either the T7 reporter plasmid, the T3 reporter plasmid, or a nonfunctional reporter plasmid. Cells were inoculated into 0.5 mL LB+antibiotics and grown to saturation overnight. The overnight cultures were diluted 1:200 into 0.15 mL LB+antibiotics+5 nM aTc and variable IPTG and grown at 37° C., 1000 rpm for 6 hours. The fluorescence of the cells was quantified using a flow cytometer (FIG. 9). Expression levels were normalized to the values obtained in the assay for FIG. 8, which used the same core fragment expression level, but only one sigma-like fragment at a time. Data shown is from three technical replicates performed on a single day.

Sequences Associated with Aspects of the Invention:

T7 RNA Polymerase
(SEQ ID NO: 1)
MNTINIAKNDFSDIELAAIPFNTLADHYGERLAREQLALEHESYEMGEAR
FRKMFERQLKAGEVADNAAAKPLITTLLPKMIARINDWFEEVKAKRGKRP
TAFQFLQEIKPEAVAYITIKTTLACLTSADNTTVQAVASAIGRAIEDEAR
FGRIRDLEAKHFKKNVEEQLNKRVGHVYKKAFMQVVEADMLSKGLLGGEA
WSSWHKEDSIHVGVRCIEMLIESTGMVSLHRQNAGVVGQDSETIELAPEY
AEAIATRAGALAGISPMFQPCVVPPKPWTGITGGGYWANGRRPLALVRTH
SKKALMRYEDVYMPEVYKAINIAQNTAWKINKKVLAVANVITKWKHCPVE
DIPAIEREELPMKPEDIDMNPEALTAWKRAAAAVYRKDKARKSRRISLEF
MLEQANKFANHKAIWFPYNMDWRGRVYAVSMFNPQGNDMTKGLLTLAKGK
PIGKEGYYWLKIHGANCAGVDKVPFPERIKFIEENHENIMACAKSPLENT
WWAEQDSPFCFLAFCFEYAGVQHHGLSYNCSLPLAFDGSCSGIQHFSAML
RDEVGGRAVNLLPSETVQDIYGIVAKKVNEILQADAINGTDNEVVTVTDE
NTGEISEKVKLGTKALAGQWLAYGVTRSVTKRSVMTLAYGSKEFGFRQQV
LEDTIQPAIDSGKGLMFTQPNQAAGYMAKLIWESVSVTVVAAVEAMNWLK
SAAKLLAAEVKDKKTGEILRKRCAVHWVTPDGFPVWQEYKKPIQTRLNLM
FLGQFRLQPTINTNKDSEIDAHKQESGIAPNFVHSQDGSHLRKTVVWAHE
KYGIESFALIHDSFGTIPADAANLFKAVRETMVDTYESCDVLADFYDQFA
DQLHESQLDKMPALPAKGNLNLRDILESDFAFA
T7 RNA Polymerase with R632S mutation
(SEQ ID NO: 2)
MNTINIAKNDFSDIELAAIPFNTLADHYGERLAREQLALEHESYEMGEAR
FRKMFERQLKAGEVADNAAAKPLITTLLPKMIARINDWFEEVKAKRGKRP
TAFQFLQEIKPEAVAYITIKTTLACLTSADNTTVQAVASAIGRAIEDEAR
FGRIRDLEAKHFKKNVEEQLNKRVGHVYKKAFMQVVEADMLSKGLLGGEA
WSSWHKEDSIHVGVRCIEMLIESTGMVSLHRQNAGVVGQDSETIELAPEY
AEAIATRAGALAGISPMFQPCVVPPKPWTGITGGGYWANGRRPLALVRTH
SKKALMRYEDVYMPEVYKAINIAQNTAWKINKKVLAVANVITKWKHCPVE
DIPAIEREELPMKPEDIDMNPEALTAWKRAAAAVYRKDKARKSRRISLEF
MLEQANKFANHKAIWFPYNMDWRGRVYAVSMFNPQGNDMTKGLLTLAKGK
PIGKEGYYWLKIHGANCAGVDKVPFPERIKFIEENHENIMACAKSPLENT
WWAEQDSPFCFLAFCFEYAGVQHHGLSYNCSLPLAFDGSCSGIQHFSAML
RDEVGGRAVNLLPSETVQDIYGIVAKKVNEILQADAINGTDNEVVTVTDE
NTGEISEKVKLGTKALAGQWLAYGVTRSVTKSSVMTLAYGSKEFGFRQQV
LEDTIQPAIDSGKGLMFTQPNQAAGYMAKLIWESVSVTVVAAVEAMNWLK
SAAKLLAAEVKDKKTGEILRKRCAVHWVTPDGFPVWQEYKKPIQTRLNLM
FLGQFRLQPTINTNKDSEIDAHKQESGIAPNFVHSQDGSHLRKTVVWAHE
KYGIESFALIHDSFGTIPADAANLFKAVRETMVDTYESCDVLADFYDQFA
DQLHESQLDKMPALPAKGNLNLRDILESDFAFA
Core fragment with 601 split
(SEQ ID NO: 3)
MNTINIAKNDFSDIELAAIPFNTLADHYGERLAREQLALEHESYEMGEAR
FRKMFERQLKAGEVADNAAAKPLITTLLPKMIARINDWFEEVKAKRGKRP
TAFQFLQEIKPEAVAYITIKTTLACLTSADNTTVQAVASAIGRAIEDEAR
FGRIRDLEAKHFKKNVEEQLNKRVGHVYKKAFMQVVEADMLSKGLLGGEA
WSSWHKEDSIHVGVRCIEMLIESTGMVSLHRQNAGVVGQDSETIELAPEY
AEAIATRAGALAGISPMFQPCVVPPKPWTGITGGGYWANGRRPLALVRTH
SKKALMRYEDVYMPEVYKAINIAQNTAWKINKKVLAVANVITKWKHCPVE
DIPAIEREELPMKPEDIDMNPEALTAWKRAAAAVYRKDKARKSRRISLEF
MLEQANKFANHKAIWFPYNMDWRGRVYAVSMFNPQGNDMTKGLLTLAKGK
PIGKEGYYWLKIHGANCAGVDKVPFPERIKFIEENHENIMACAKSPLENT
WWAEQDSPFCFLAFCFEYAGVQHHGLSYNCSLPLAFDGSCSGIQHFSAML
RDEVGGRAVNLLPSETVQDIYGIVAKKVNEILQADAINGTDNEVVTVTDE
N
Core fragment with 601 split + SZ17
(SEQ ID NO: 4)
MNTINIAKNDFSDIELAAIPFNTLADHYGERLAREQLALEHESYEMGEAR
FRKMFERQLKAGEVADNAAAKPLITTLLPKMIARINDWFEEVKAKRGKRP
TAFQFLQEIKPEAVAYITIKTTLACLTSADNTTVQAVASAIGRAIEDEAR
FGRIRDLEAKHFKKNVEEQLNKRVGHVYKKAFMQVVEADMLSKGLLGGEA
WSSWHKEDSIHVGVRCIEMLIESTGMVSLHRQNAGVVGQDSETIELAPEY
AEAIATRAGALAGISPMFQPCVVPPKPWTGITGGGYWANGRRPLALVRTH
SKKALMRYEDVYMPEVYKAINIAQNTAWKINKKVLAVANVITKWKHCPVE
DIPAIEREELPMKPEDIDMNPEALTAWKRAAAAVYRKDKARKSRRISLEF
MLEQANKFANHKAIWFPYNMDWRGRVYAVSMFNPQGNDMTKGLLTLAKGK
PIGKEGYYWLKIHGANCAGVDKVPFPERIKFIEENHENIMACAKSPLENT
WWAEQDSPFCFLAFCFEYAGVQHHGLSYNCSLPLAFDGSCSGIQHFSAML
RDEVGGRAVNLLPSETVQDIYGIVAKKVNEILQADAINGTDNEVVTVTDE
NGGSGGGSNEKEELKSKKAELRNRIEQLKQKREQLKQKIANLRKEIEAY
T7 sigma-like fragment with 601 split
(SEQ ID NO: 5)
NTGEISEKVKLGTKALAGQWLAYGVTRSVTKSSVMTLAYGSKEFGFRQQV
LEDTIQPAIDSGKGLMFTQPNQAAGYMAKLIWESVSVTVVAAVEAMNWLK
SAAKLLAAEVKDKKTGEILRKRCAVHWVTPDGFPVWQEYKKPIQTRLNLM
FLGQFRLQPTINTNKDSEIDAHKQESGIAPNFVHSQDGSHLRKTVVWAHE
KYGIESFALIHDSFGTIPADAANLFKAVRETMVDTYESCDVLADFYDQFA
DQLHESQLDKMPALPAKGNLNLRDILESDFAFA
T7 sigma-like fragment with 601 split + SZ18
(SEQ ID NO: 6)
MSIAATLENDLARLENENARLEKDIANLERDLAKLEREEAYFGGSGGKNT
GEISEKVKLGTKALAGQWLAYGVTRSVTKSSVMTLAYGSKEFGFRQQVLE
DTIQPAIDSGKGLMFTQPNQAAGYMAKLIWESVSVTVVAAVEAMNWLKSA
AKLLAAEVKDKKTGEILRKRCAVHWVTPDGFPVWQEYKKPIQTRLNLMFL
GQFRLQPTINTNKDSEIDAHKQESGIAPNFVHSQDGSHLRKTVVWAHEKY
GIESFALIHDSFGTIPADAANLFKAVRETMVDTYESCDVLADFYDQFADQ
LHESQLDKMPALPAKGNLNLRDILESDFAFA
T3 sigma-like fragment with 601 split + SZ18
(SEQ ID NO: 7)
MSIAATLENDLARLENENARLEKDIANLERDLAKLEREEAYFGGSGGKNT
GEISEKVKLGTKALAGQWLAYGVTRSVTKRSVMTLAYGSKEFGFRQQVLE
DTIQPAIDSGKGLMFTQPNQAAGYMAKLIWESVSVTVVAAVEAMNWLKSA
AKLLAAEVKDKKTGEILRKRCAVHWVTPDGFPVWQEYKKPIQKRLDMIFL
GQFRLQPTINTNKDSEIDAHKQESGIAPNFVHSQDGSHLRKTVVWAHEKY
GIESFALIHDSFGTIPADAANLFKAVRETMVDTYESCDVLADFYDQFADQ
LHESQLDKMPALPAKGNLNLRDILESDFAFA
K1FR sigma-like fragment with 601 split + SZ18
(SEQ ID NO: 8)
MSIAATLENDLARLENENARLEKDIANLERDLAKLEREEAYFGGSGGKNT
GEISEKVKLGTKALAGQWLAYGVTRSVTKRSVMTLAYGSKEFGFRQQVLE
DTIQPAIDSGKGLMFTQPNQAAGYMAKLIWESVSVTVVAAVEAMNWLKSA
AKLLAAEVKDKKTGEILRKRCAVHWVTPDGFPVWQEYKKPIQTRLNLRFL
GSFNLQPTVNTNKDSEIDAHKQESGIAPNFVHSQDGSHLRKTVVWAHEKY
GIESFALIHDSFGTIPADAANLFKAVRETMVDTYESCDVLADFYDQFADQ
LHESQLDKMPALPAKGNLNLRDILESDFAFA
Synzip 17
(SEQ ID NO: 9)
NEKEELKSKKAELRNRIEQLKQKREQLKQKIANLRKEIEAYK
Synzip 18
(SEQ ID NO: 10)
SIAATLENDLARLENENARLEKDIANLERDLAKLEREEAYF
T7 sigma-like fragment with 601 split with Met,
Xaa
(SEQ ID NO: 11)
MXNTGEISEKVKLGTKALAGQWLAYGVTRSVTKSSVMTLAYGSKEFGFRQ
QVLEDTIQPAIDSGKGLMFTQPNQAAGYMAKLIWESVSVTVVAAVEAMNW
LKSAAKLLAAEVKDKKTGEILRKRCAVHWVTPDGFPVWQEYKKPIQTRLN
LMFLGQFRLQPTINTNKDSEIDAHKQESGIAPNFVHSQDGSHLRKTVVWA
HEKYGIESFALIHDSFGTIPADAANLFKAVRETMVDTYESCDVLADFYDQ
FADQLHESQLDKMPALPAKGNLNLRDILESDFAFA
T7 sigma-like fragment with 601 split with Met,
Lys
(SEQ ID NO: 12)
MKNTGEISEKVKLGTKALAGQWLAYGVTRSVTKSSVMTLAYGSKEFGFRQ
QVLEDTIQPAIDSGKGLMFTQPNQAAGYMAKLIWESVSVTVVAAVEAMNW
LKSAAKLLAAEVKDKKTGEILRKRCAVHWVTPDGFPVWQEYKKPIQTRLN
LMFLGQFRLQPTINTNKDSEIDAHKQESGIAPNFVHSQDGSHLRKTVVWA
HEKYGIESFALIHDSFGTIPADAANLFKAVRETMVDTYESCDVLADFYDQ
FADQLHESQLDKMPALPAKGNLNLRDILESDFAFA

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

What is claimed is:

1. A recombinant T7 RNA polymerase comprising
a core fragment that has no RNA polymerase activity by itself and no ability to bind and/or target a promoter DNA sequence; and
a sigma-like fragment that has specificity for a promoter DNA sequence, but comprises no RNA polymerase activity,
wherein the sigma-like fragment binds the core fragment to form a protein complex that has RNA polymerase activity, targets a promoter DNA sequence for which the sigma-like fragment has specificity and initiates transcription of RNA from the promoter DNA sequence, and
wherein the sigma-like fragment does not initiate transcription of RNA without binding to the core fragment.

2. The recombinant T7 polymerase of claim 1, wherein the T7 RNA polymerase is split at an amino acid selected from the group consisting of amino acids 67-74, 160-206, 301-302, 564-607, and 763-770 of T7 RNA polymerase into an N-terminal fragment and a C-terminal fragment, optionally wherein the T7 RNA polymerase is split at an amino acid selected from the group consisting of amino acids 67, 179, 301, 601 and 767 of T7 RNA polymerase.

3. (canceled)

4. (canceled)

5. The recombinant T7 polymerase of claim 1, wherein a methionine residue is added to the N-terminus of the C-terminal fragment, and optionally wherein one or more variable amino acid residues and/or one or more amino acid residues from the N-terminal fragment are added to the C-terminal fragment, optionally wherein the T7 polymerase is split at amino acid 601 to yield (1) a core fragment consisting of amino acids 1-601 of T7 polymerase (1:601) and (2) a sigma-like fragment consisting of a dipeptide of methionine and a variable amino acid joined to amino acids 601-883 of T7 polymerase (M X 601:883), optionally a dipeptide of methionine and a lysine joined to amino acids 601-883 of T7 polymerase (M K 601:883).

6. (canceled)

7. The recombinant T7 polymerase of claim 1, wherein the core fragment and the sigma-like fragments are each fused to heterospecific protein interaction domains (PID) that interact with each other, to form a PID-core fragment fusion and PID-sigma-like fragment fusions, and wherein the association of the core fragment and the sigma-like fragment to form the recombinant T7 polymerase is increased relative to the association of the core fragment and the sigma-like fragment without fusion to PIDs,
optionally wherein the PIDs are coiled-coil domains,
optionally wherein the coiled-coil domains are synzip coiled-coil domains, optionally wherein the coiled-coil domains are synzip coiled-coil domains synzip 17 and synzip 18.

8.-10. (canceled)

11. The recombinant T7 polymerase of claim 1, wherein a flexible linker links the PIDs to the core fragment or the sigma-like fragment, optionally wherein the flexible linkers comprise amino acids, optionally wherein the flexible linkers comprise 5-7 amino acids.

12. (canceled)

13. (canceled)

14. The recombinant T7 polymerase of claim 1, wherein the sigma-like fragment of the recombinant T7 RNA polymerase is engineered to have a non-native promoter DNA sequence specificity.

15. A system comprising
a core fragment that has no RNA polymerase activity by itself and no ability to bind and/or target a promoter DNA sequence; and
a set of sigma-like fragments, each of which has specificity for and/or targets a promoter DNA sequence but has no RNA polymerase activity by itself;
wherein each sigma-like fragment in the set of sigma-like fragments binds the core fragment to form a protein complex that has RNA polymerase activity, targets a promoter DNA sequence for which the sigma-like fragment has specificity and initiates transcription of RNA from the promoter DNA sequence, and
wherein the sigma-like fragments do not initiate transcription of RNA without binding to the core fragment.

16. The system of claim 15, wherein the core fragment and each of the set of sigma-like fragments is a fragment of T7 RNA polymerase.

17. The system of claim 16, wherein the T7 RNA polymerase is split at an amino acid selected from the group consisting of amino acids 67-74, 160-206, 301-302, 564-607, and 763-770 of T7 RNA polymerase into an N-terminal fragment and a C-terminal fragment, optionally wherein the T7 RNA polymerase is split at an amino acid selected from the group consisting of amino acids 67, 179, 301, 601 and 767 of T7 RNA polymerase.

18. (canceled)

19. (canceled)

20. The system of claim 15, wherein a methionine residue is added to the N-terminus of the C-terminal fragment, and optionally wherein one or more variable amino acid residues and/or one or more amino acid residues from the N-terminal fragment are added to the C-terminal fragment, optionally wherein the T7 polymerase is split at amino acid 601 to yield (1) a core fragment consisting of amino acids 1-601 of T7 polymerase (1:601) and (2) a sigma-like fragment consisting of a dipeptide of methionine and a variable amino acid joined to amino acids 601-883 of T7 polymerase (M X 601:883), optionally a dipeptide of methionine and a lysine joined to amino acids 601-883 of T7 polymerase (M K 601:883).

21. (canceled)

22. The system of claim 15, wherein the core fragment and the sigma-like fragments are each fused to heterospecific protein interaction domains (PID) that interact with each other, to form a PID-core fragment fusion and PID-sigma-like fragment fusions, and wherein the association of the core fragment and the sigma-like fragments to form the protein complex is increased relative to the association of the core fragment and the sigma-like fragments without fusion to PIDs,
optionally wherein the PIDs are coiled-coil domains,
optionally wherein the coiled-coil domains are synzip coiled-coil domains, optionally wherein the coiled-coil domains are synzip coiled-coil domains synzip 17 and synzip 18.

23.-25. (canceled)

26. The system of claim 22, wherein a flexible linker links the PIDs to the core fragment and/or the sigma-like fragments,
optionally wherein the flexible linkers comprise amino acids, optionally wherein the flexible linkers comprise 5-7 amino acids.

27. (canceled)

28. (canceled)

29. The system of claim 15, wherein each of the set of sigma-like fragments is engineered to have a different promoter DNA sequence specificity.

30. The system of claim 15, further comprising nucleic acids comprising promoter DNA sequences that are specifically bound by each of the set of sigma-like fragments, optionally wherein each promoter is activated at least 10-fold more by its cognate sigma-like factor than by any non-cognate sigma-like factor.

31. (canceled)

32. The system of claim 15, wherein the promoter DNA sequences are operably linked to a reporter sequence and/or a protein coding sequence.

33. The system of claim 15, wherein the core fragment and each of the set of sigma-like fragments is independently expressed, optionally wherein the core fragment is expressed constitutively from a single copy plasmid and/or wherein each sigma-like fragment is expressed from a medium-high copy plasmid.

34. (canceled)

35. The system of claim 15, wherein expression of at least each of the set of sigma-like fragments is controlled by inputs to the system, optionally conditions that the system is exposed to.

36. The system of claim 15, wherein expression of the core fragment is constitutive.

37. The system of claim 15, wherein the system is in a cell.

38. A method of controlling RNA transcription of one or more DNA sequences comprising placing the one or more DNA sequences under the transcriptional control of the system of claim 15,
optionally wherein each of the one or more DNA sequences is operably linked to a promoter DNA sequence that is specifically bound by at least one of the set of sigma-like fragments,
optionally wherein the ratio of the expression of the set of variable proteins determines output of the system.

39. (canceled)

40. (canceled)


SEQUENCE LISTING (12 sequences)

SEQ ID NO: 1 - protein, 883 aa, Bacteriophage T7
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 2 - protein, 883 aa, Artificial Sequence
  Synthetic Polypeptide
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKSSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 3 - protein, 601 aa, Artificial Sequence
  Synthetic Polypeptide
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDEN

SEQ ID NO: 4 - protein, 649 aa, Artificial Sequence
  Synthetic Polypeptide
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENGGSGGGSNEKEELKSKKAELRNRIEQLKQKREQLKQKIANLRKEIEAY

SEQ ID NO: 5 - protein, 283 aa, Artificial Sequence
  Synthetic Polypeptide
NTGEISEKVKLGTKALAGQWLAYGVTRSVTKSSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 6 - protein, 331 aa, Artificial Sequence
  Synthetic Polypeptide
MSIAATLENDLARLENENARLEKDIANLERDLAKLEREEAYFGGSGGKNTGEISEKVKLGTKALAGQWLAYGVTRSVTKSSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 7 - protein, 331 aa, Artificial Sequence
  Synthetic Polypeptide
MSIAATLENDLARLENENARLEKDIANLERDLAKLEREEAYFGGSGGKNTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQKRLDMIFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 8 - protein, 331 aa, Artificial Sequence
  Synthetic Polypeptide
MSIAATLENDLARLENENARLEKDIANLERDLAKLEREEAYFGGSGGKNTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLRFLGSFNLQPTVNTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 9 - protein, 42 aa, Artificial Sequence
  Synthetic Polypeptide
NEKEELKSKKAELRNRIEQLKQKREQLKQKIANLRKEIEAYK

SEQ ID NO: 10 - protein, 41 aa, Artificial Sequence
  Synthetic Polypeptide
SIAATLENDLARLENENARLEKDIANLERDLAKLEREEAYF

SEQ ID NO: 11 - protein, 285 aa, Artificial Sequence
  Synthetic Polypeptide
MXNTGEISEKVKLGTKALAGQWLAYGVTRSVTKSSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 12 - protein, 285 aa, Artificial Sequence
  Synthetic Polypeptide
MKNTGEISEKVKLGTKALAGQWLAYGVTRSVTKSSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA